(19) United States
(12) Patent Application Publication
(10) Pub. No.: US 2025/0288789 A1
(43) Pub. Date: Sep. 18, 2025

(54) Safe Disconnect Coupler for Urinary Catheter

(71) Applicant: Yankee Medical Inc., San Francisco, CA (US)

(72) Inventor: Nicholas E. Scianmarello, San Francisco, CA (US)

(73) Assignee: Yankee Medical Inc., San Francisco, CA (US)

(21) Appl. No.: 19/069,389

(22) Filed: Mar. 4, 2025

Related U.S. Application Data
(60) Provisional application No. 63/564,860, filed on Mar. 13, 2024.

(51) Int. Cl. A61M 39/10; A61M 25/00; A61M 25/10

(52) U.S. Cl. CPC A61M 39/10; A61M 25/0017; A61M 25/10182; A61M 25/10185; A61M 2039/1061; A61M 2207/00

(57) ABSTRACT

A safe disconnect coupler for medical tubing includes a coupler body that mates with a stalk, the coupler body having protruding cylindrical male nose portion that mates into a receptacle of the stalk. A resilient O-ring around a base of the nose mates into a circumferential detent groove in the receptacle. The O-ring and detent groove are shaped such that the nose and stalk are held slightly apart, and a small, calibrated (e.g., 1 N) tensile force is needed to pull them apart. However, when a bending force is applied to the coupler assembly, the nose bears against the inside of the stalk, keeping the two aligned such the bending force alone will not pry them apart. For a Foley catheter, an additional adapter can open a balloon injection port when pulled, and/or the coupler can be kitted with the drain bag.

### CROSS-REFERENCES TO RELATED APPLICATIONS

This application claims the benefit of U.S. Provisional Application No. 63/564,860, filed 13 Mar. 2024, which is hereby incorporated by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### BACKGROUND

### 1. Field of the Art

Embodiments of the present invention generally relate to break-apart tubing connectors or couplings for urinary or other drainage or non-drainage catheters. In particular embodiments, the break apart connector operates in conjunction with a valve or other mechanism that subsequently allows a Foley catheter balloon to deflate.

### 2. Description of the Related Art

Foley catheter systems are common medical devices used to provide real time urine drainage and output information. A urinary Foley catheter is an indwelling tubular structure that extends from inside a patient's bladder outside of the patient to a drainage bag. For a male, a catheter extends through the urethra of a penis; for a female, it extends through the urethra. The catheter allows drainage of urine if a patient is unable to void volitionally due to medical or anatomical reasons.

The urinary catheter is maintained in the bladder with a balloon filled with distilled or deionized water. The liquid comes through a balloon fill port once the balloon is set in place. The (American Society for Testing and Materials) ASTM F623 Standard Performance Specifications for Foley Catheters require that the entire balloon of catheters, labeled French size 14 through 26, should not pass into or through the funnel barrel with a size of 28 Fr. It requires the maintenance of a traction force of 0.45 kg (1 lb.).

Ten to twenty-five percent of all hospitalized patients typically require urinary catheterization. Ninety percent of patients in the intensive care unit of any hospital will typically require catheterization. A large proportion of patients undergoing surgery under general and spinal anesthesia will require urinary catheterization.

There are several complications that are associated with urinary catheterization including infection, pain, and traumatic urethral damage. Up to three percent of patients that have indwelling urinary catheterization will experience some type of urethral trauma, although the incidence is likely underreported. A common source of urethral trauma associated with urinary catheterization is inadvertent traction on urinary catheters, or traumatic urinary catheter pull out. This occurs when a patient is medically sedated, agitated, or the catheter or the drainage tubing is simply caught on something when a patient moves or ambulates. The inflated balloon is pulled into the narrow, delicate urethra and often completely through the entire urethra, causing tearing, bleeding, and other tissue damage. Short term complications include acute infection, excruciating pain, and urethral bleeding. The damage to the fragile tissue of the urethra can lead to longer term complications including urinary incontinence or urethral obstruction due to scar formation, strictures, that can require complex surgical repair.

The length of the catheter outside of the urethra can be made relatively short, for example 15-20 cm, and relatively easy to secure, such as with a CATH-SECURE® device (MC Johnson Company, Fort Myers, Florida, United States of America) that anchors the catheter hub of the device to the patient's leg. In contrast, the length of tubing to the catheter drainage bag, sometimes upwards of 125 cm, is difficult to completely secure. This is despite urine drainage bags also commonly being secured to the patient's leg via straps. The length of the tubing to the drainage bag makes it prone to catch on crevices of hospital beds and furniture. It is also a tempting target to be grabbed by an agitated or delirious patient. Because the urine drainage bag is strongly anchored to the patient, any snag or pull on the tubing will exert force at the other end-transmitted through the catheter to the balloon inside the patient.

There is a need in the art for safety devices that can mitigate traumatic injuries from Foley catheter pullout events.

### BRIEF SUMMARY

Generally, a medical tubing coupler is described that uses an O-ring seated in a depression calibrated to give way at or above a predetermined tensile force and paired with a loose male-female slip fitting that prevents a large non-tensile, shear force from levering the O-ring out of its seat. The O-ring is geometrically sized to protrude enough from the male fitting to maintain its seal even when the male-female parts are bent to their maximum extents against each other.

For Foley catheters, the coupler can have an additional adapter that connects to the Foley catheter balloon filling port and opens its drain promptly after the coupler gives way. The adapter is in the form of a male luer, and it attaches within a female injection port.

The coupler can be attached directly to or kitted with a urinary bag, therefore falling under lower risk medical device classifications.

Some embodiments of the invention are related to a safe disconnect coupler apparatus for medical tubing, the apparatus including a coupler body with a longitudinal axis and a lumen extending through a first tubing connector of the coupler body and through a cylindrical nose of the coupler body, a resilient O-ring circumferentially surrounding a base of the nose and protruding radially from the nose, and a slip-off stalk with a longitudinal axis and a lumen extending through a second tubing connector of the stalk and through a cylindrical cavity of the stalk, the cavity having a detent groove extending circumferentially around an inside wall of the cavity, wherein the coupler body and the stalk are configured to mate together by inserting the nose into the cavity such that the O-ring protrudes into and seals against the detent groove while maintaining a gap between the nose and the inside wall, wherein when misalignment forces are applied to the mated coupler body and the stalk, a tip of the coupler body nose bears against the inside wall of the stalk to preserve alignment between the mated coupler body and the stalk, and the O-ring continues to protrude into and seal against the detent groove all of a way around a circumference of the cavity.

The O-ring protrusion, a material of the O-ring, and a shape of the detent groove can all be selected such that a mated coupler body and stalk are configured to separate upon a longitudinal pull-apart force between 1 N and 50 N. Given a distance D from the tip of the coupler body nose to the O-ring, the O-ring can protrude from the coupler body by at least D times) tan (5° when uncompressed. The cylindrical nose and the cylindrical cavity can be slightly conical frustums.

The O-ring can be a polymer that is over-molded around the nose. The polymer can be selected from the group consisting of a silicone, a fluoroelastomer, an acrylonitrile butadiene rubber, a polypropylene, and a polyethylene. In some embodiments, the O-ring is a distinct part that is held within a trench seat in the nose. The O-ring can be a first O-ring, and the apparatus can further include a second O-ring on the nose and configured to seal against the inside wall. A lubricant or lubricious polymer can be applied to the O-ring.

The coupler body can include a rounded reducer section and an outer profile with no hooked elements or sharp protrusions. There can exist a sample port in the coupler body, the sample port comprising a septum configured to allow needle access inside the coupler body.

The apparatus can further include a male luer adapter having a fluid path therethrough and a first end configured to mate with a female balloon injection port of a catheter balloon and keep open a valve in the injection port, and a removable stopper on a second end of the male luer adapter, the stopper connected with the coupler body, wherein the stopper is configured to seal the fluid path until pulled away from the male luer adapter by force transmitted from the coupler body. The apparatus can further include a flexible tether connecting the removable stopper to the coupler body. The removable stopper can be rigidly connected with the coupler body.

The nose of the coupler body can be mated with the cavity of the stalk, as an assembled device. The apparatus can further include a reconnection prevention mechanism configured to prevent the coupler body and cavity from being mated again after the coupler body and cavity are pulled apart.

A urine collection vessel, a catheter hub, and the safe disconnect coupler apparatus can be kitted together.

Some embodiments are related to a method of manufacturing a safe disconnect coupler for medical tubing, the method including providing a coupler body with a longitudinal axis and a lumen extending through a first tubing connector of the coupler body and through a cylindrical nose of the coupler body, forming or affixing a resilient O-ring circumferentially around a base of the nose such that the O-ring protrudes radially from the nose, providing a slip-off stalk with a longitudinal axis and a lumen extending through a second tubing connector of the stalk and through a cylindrical cavity of the stalk, the cavity having a detent groove extending circumferentially around an inside wall of the cavity, and mating together the coupler body with the stalk by inserting the nose into the cavity such that the O-ring protrudes into and seals against the detent groove while maintaining a gap between the nose and the inside wall.

The method can further include flexing the mated coupler body and stalk such that a tip of the coupler body nose bears against the inside wall to preserve alignment between the mated coupler body and the stalk, and such that the O-ring continues to protrude into and seal against the detent groove all of the way around a circumference of the cavity. The method can include testing the assembled device by pulling apart the mated coupler body and stalk with a longitudinal force between 1 N and 50 N.

Some embodiments are related to a method of using a safe disconnect coupler for medical tubing, the method including providing a coupler body with a longitudinal axis and a lumen extending through a first tubing connector of the coupler body and through a cylindrical nose of the coupler body, the coupler having a resilient O-ring affixed circumferentially around a base of the nose such that the O-ring protrudes radially from the nose, providing a slip-off stalk with a longitudinal axis and a lumen extending through a second tubing connector of the stalk and through a cylindrical cavity of the stalk, the cavity having a detent groove extending circumferentially around an inside wall of the cavity, wherein the coupler body is mated with the stalk in that the nose projects into the cavity such that the O-ring protrudes into and seals against the detent groove while maintaining a gap between the nose and the inside wall, connecting medical tubing from a urine collection vessel to one of the tubing connectors, and connecting a urinary catheter to the other one of the tubing connectors.

The method can further include flexing the mated coupler body and stalk such that a tip of the coupler body nose bears against the inside wall to preserve alignment between the mated coupler body and the stalk, and such that the O-ring continues to protrude into and seal against the detent groove all of a way around a circumference of the cavity. The method can include pulling apart the mated coupler body and stalk with a longitudinal force between 1 N and 50 N.

The method can include providing a male luer adapter having a fluid path therethrough, providing a removable stopper on a second end of the male luer adapter, the stopper connected with the coupler body, and pushing the male luer adapter into a female balloon injection port for a catheter balloon to open a valve in the injection port.

The method can include pulling apart the mated coupler body and stalk with a longitudinal force between 1 N and 50 N, and then pulling away the stopper from the male luer using force transmitted from the coupler body, the male luer keeping the injection port valve open and allowing a catheter balloon to drain and deflate.

Some embodiments are related to a safe disconnect urinary catheter collection vessel apparatus including a urine collection vessel, a medical tubing connected with the vessel, and a safe disconnect coupler connected to an end of the medical tubing, the coupler being configured to separate into a coupler body and another part upon a longitudinal pull-apart force between 1 N and 50 N.

The apparatus can further include a male luer adapter having a fluid path therethrough and a first end configured to mate with a female balloon injection port of a catheter balloon and keep open a valve in the injection port, and a removable stopper on a second end of the male luer adapter, the stopper connected with the coupler body, wherein the stopper is configured to seal the fluid path until pulled away from the male luer adapter by force transmitted from the coupler body. The apparatus can include a flexible tether connecting the removable stopper to the coupler body. The removable stopper can be rigidly connected with the coupler body. A female balloon injection port can be mated with the male luer adapter. The apparatus can include a urinary catheter connected with the coupler.

### BRIEF DESCRIPTION OF THE DRAWINGS

FIG. 1 is a perspective view of an assembled safe disconnect coupler in accordance with an embodiment.

FIG. 2 is a perspective view of a coupler body of the coupler of FIG. 1.

FIG. 3 is a perspective view of a hollow stalk of the coupler of FIG. 1.

FIG. 4 is a longitudinal cross-section of the assembled coupler of FIG. 1.

FIG. 5A is a longitudinal cross-section of an assembled coupler being bent such that its coupler body and stalk are subject to misalignment forces in accordance with an embodiment.

FIG. 5B is a close-up cross-section of a coupler body nose bearing against an inside wall of the stalk given the misalignment forces of FIG. 5A.

FIG. 6 is a longitudinal cross-section of an unmated coupler body in accordance with an embodiment.

FIG. 7 is a side view of the coupler undergoing bending forces of FIG. 5A.

FIG. 8 is a longitudinal cross-section of a coupler with both a force-calibrated O-ring and a face-sealing O-ring in accordance with an embodiment.

FIG. 9 is a longitudinal cross-section of a coupler with a rigidly connected balloon injection port holster in accordance with an embodiment.

FIG. 10 is a longitudinal cross-section of a coupler with a tethered balloon injection port holster in accordance with an embodiment.

FIG. 11 is a longitudinal cross-section of a plugged male luer in accordance with an embodiment.

FIG. 12 is a longitudinal cross-section of a burst plate male luer in accordance with an embodiment.

FIG. 13 illustrates a side view of a coupler reconnection prevention mechanism in accordance with an embodiment.

FIG. 14 illustrates a urine drainage bag ready to be connected to a coupler in accordance with an embodiment.

FIG. 15 is a top view of a coupler body in accordance with an embodiment.

FIG. 16 is a bottom view of a coupler body in accordance with an embodiment.

FIG. 17 is a left side view of a coupler body in accordance with an embodiment.

FIG. 18 is a right side view of a coupler body in accordance with an embodiment.

FIG. 19 is a front view of a coupler body in accordance with an embodiment.

FIG. 20 is a rear view of a coupler body in accordance with an embodiment.

FIG. 21 is a flowchart illustrating a process in accordance with an embodiment.

FIG. 22 is a flowchart illustrating a process in accordance with an embodiment.

### DETAILED DESCRIPTION

Although the break-apart coupler may be used with many different kinds of medical tubing, from feeding tubes to dialysis machines, most examples herein will be focused on use with drainage tubing for indwelling urinary catheters.

A purpose of the urinary catheter safety device is to prevent traumatic urinary catheter pull out during inadvertent or dangerous traction. The safe disconnector mechanism, which can sit between the urinary catheter and drainage bag, maintains a necessary 0.45 kg (1-lb) weight force of traction of the ASTM F623 standard but releases and completely disconnects at a very small increment above this force.

The safe disconnect device can be tailored to disconnect at 50 newtons (N), 40 N, 30 N, 25 N, 20 N, or 10 N, among other force strengths.

Such a device can maintain the integrity of the drainage system while still facilitating the safe disconnection of the drainage bag before significant urethral trauma can occur.

The device can be a coupler for two medical tubes, the coupler being able to split apart into two sections, a coupler body and a stalk. The coupler body has a male portion that loosely slip-fits into a female portion of the stalk, and an O-ring between them is calibrated to allow them to separate above a specified tensile force. If one of the two parts is yanked to the side, i.e., not in a tensile direction, then the male and female portions bear together to keep the O-ring sealed. The slip fit portions can have slick surfaces so that when the component of tensile force is above the minimum amount, the coupler splits apart even when the male and female portions bear together.

A second adapter, which mates with the Foley catheter balloon port, can rapidly open the port and deflate the catheter balloon to mitigate self-harm to the patient. A new catheter can be placed once the patient is stabilized.

A “cylindrical” element includes an element that is generally circular or oval in cross sections along a longitudinal axis, or as otherwise known in the art. This includes geometrically right cylinders as well as cylinders that taper and conical frustums.

A “slightly conical frustum” includes a conical frustum with an angle between its longitudinal axis and side that is less-than-or-equal-to 30°, 20°, 15°, 12.5°, 10°, 7.5°, 5°, 4°, 3°, 2°, or 1°, or as otherwise known in the art.

A “lubricant” can include oil, gel, powder, or other lubricating material, or as otherwise known in the art. For example, talcum powder can be used as a lubricant.

A urine collection “vessel” can include a bag, sealed or otherwise, a bed pan, or other vessel for storing urine or other liquid waste, or as otherwise known in the art.

A sample port with a septum that allows needle access can include an area with an elastomeric septum within a recess, or as otherwise known in the art. The septum can also be a tube septum, or a plug can be used.

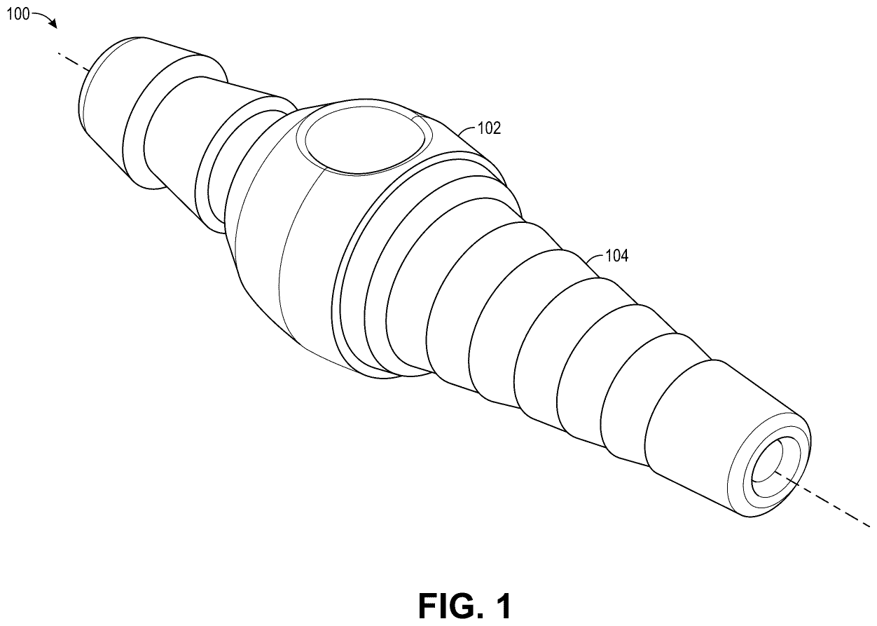

FIG. 1 is a perspective view of an assembled safe disconnect coupler 100. Coupler 100 is assembled from two major parts: coupler body 102 and stalk 104.

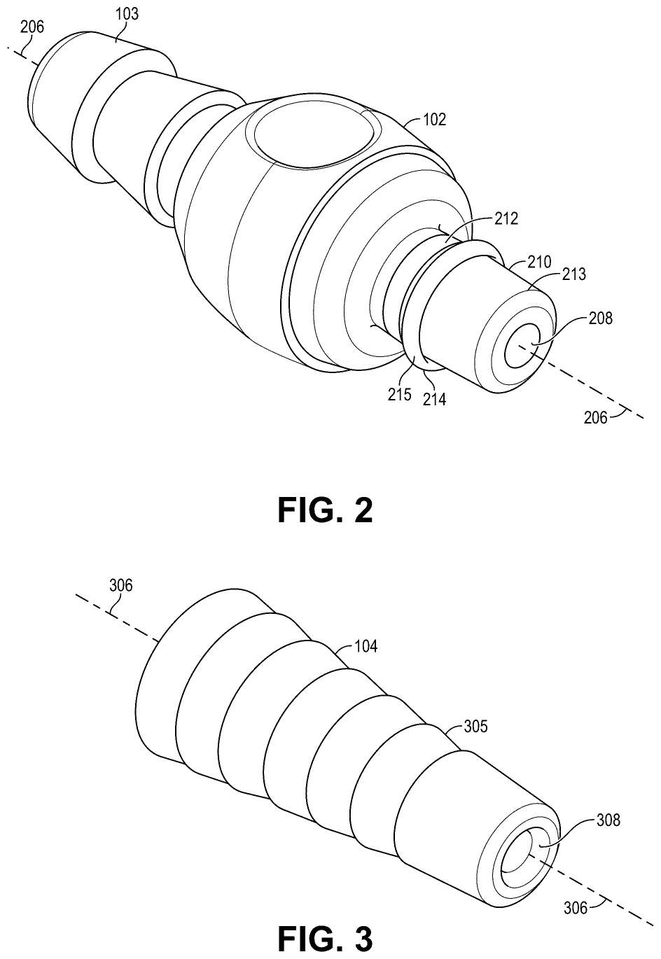

FIG. 2 illustrates coupler body 102, which has longitudinal axis 206 running lengthwise through it. On one end of coupler body 102 is medical tubing connector 103, for connecting externally with medical-grade tubing. On the other end of coupler body 102 is cylindrical nose 210. Lumen 208 extends all of the way through coupler body 102, including through tubing connector 103 and nose 210, symmetrically around longitudinal axis 206.

Nose 210 is connected to the rest of coupler body 102 through nose base 212, and it terminates at a distal end with nose tip 213. Base 212 and tip 213 have slick plastic surfaces in order to facilitate sliding away from a connected slip-off stalk (not shown in figure).

O-ring 214 surrounds base 212 around the base's circumference. It protrudes radially, i.e., in directions perpendicular to longitudinal axis 206, from the nose, its protrusion being the material above the lip of a trench seat in which O-ring 214 is tightly secured.

The O-ring can be composed of resilient material, such as resilient polymers, including silicone, fluoroelastomer, acrylonitrile butadiene rubber, polypropylene, and polyethylene. In the exemplary embodiment, O-ring 214 is shown as its own, distinct part that is separately molded then slipped into a trench seat molded into nose 210. In other embodiments, the O-ring is a polymer or other resilient material that is overmolded circumferentially around the nose.

In the exemplary embodiment, lubricant or lubricious polymer 215 is applied to O-ring 214 in order to better normalize friction, and therefore necessary pull off forces. That is, the lubrication can lower the pull-off force tolerances such that the necessary tensile force to pull apart an assembled device is nearer a desired minimum value.

FIG. 3 illustrates hollow stalk 104, which has longitudinal axis 306 running lengthwise through it. When mated with coupler body 102 (FIG. 2), longitudinal axis 206 of the coupler body aligns with longitudinal axis 306 of the stalk.

On one end of stalk 104 is medical tubing connector 305, and on the other side is a cavity in which the nose of the coupler body can be inserted. Lumen 308 extends all the way through stalk 104, including through tubing connector 105 and the cavity, symmetrically around longitudinal axis 306.

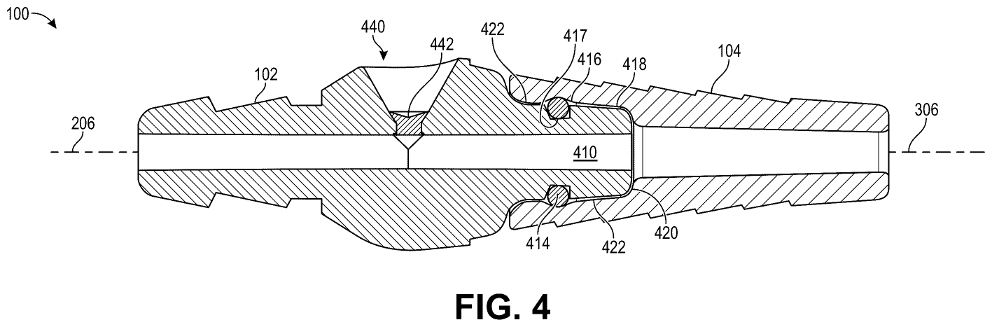

FIG. 4 is a longitudinal cross-section of assembled coupler 100, showing coupler body 102 mated with stalk 104. Coupler body nose 410 is inserted into stalk cavity 420. Meanwhile, O-ring 414, which sits firmly attached to node 410 in trench seat 417 at the base of the nose, protrudes into detent groove 416. Detent groove 416 extends circumferentially around inside wall 418 of cylindrical cavity 420.

Sample port 440 is in coupler body 102. In the exemplary embodiment it is shown with a conical recessed entrance so as to direct a wayward needle to its center. In the center of port 440 is septum 442. Septum 442 is made of a resilient material under compression that when punctured by a needle will self-seal. The resilient material may be a tube septum, plug, and/or the same material used for the O-ring.

The sample port is recessed in the coupler body and has no hooked elements or sharp protrusions.

Gap 422 between nose 410 and inside wall 418 of the cavity is maintained by pressure from O-ring 414 exerted against the inside wall, holding much of it slightly away from the surface of the nose. The size of and positions of gap 422 can change as relatively small forces wiggle coupler body 102 or stalk 104 laterally with respect to each other. Larger lateral forces may change the gap considerably.

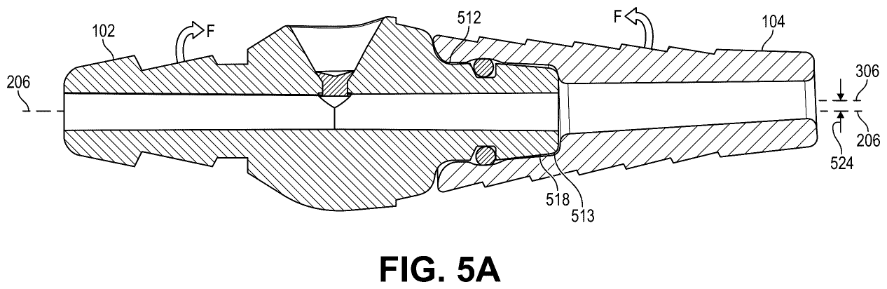

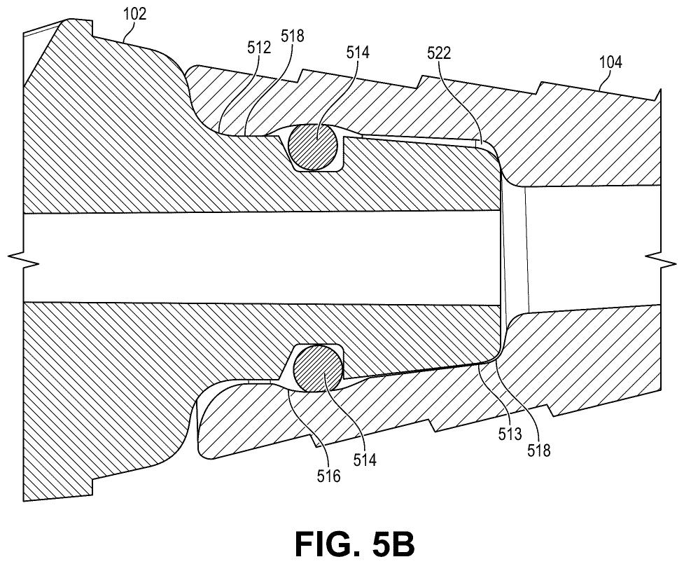

FIG. 5A is a longitudinal cross-section showing the assembled coupler subject to large misalignment forces, such as when its ends are torqued against each other. Specifically, external forces F result in slight rotations, with coupler body 102 rotated clockwise and stalk 104 rotated counterclockwise. With such rotations, longitudinal axis 206 of coupler body 102 misaligns from longitudinal axis 306 of stalk 104. Angle 524 between longitudinal axes 206 and 306 may be referred to as a misalignment angle.

At a certain misalignment force, nose tip 513 of the male nose hits against inside wall 518 of the stalk cavity. Nose base 512 of the male nose also hits against inside wall 518. These bearing spots keep coupler body 102 and stalk 104 aligned, at least to a maximum angle 524 as in the figure.

FIG. 5B is a close-up cross-section of the bearing areas of coupler body 102 and stalk 104. In the upper left, base 512 of the nose bears against inside wall 518, while in the upper right gap 522 widens. Meanwhile, in the lower right, tip 513 of the nose bears against another part of inside wall 518. In the lower left, the gap widens. The bearings trap the nose from rotating more with respect to the cavity-so that their misalignment grows no further than the allowed maximum misalignment angle.

At this maximum angle, O-ring 514 continues to protrude into, squeeze against, and seal against detent groove 516 all of the way around its circumference and the inner circumference of the cavity. Because the elongated nose and cavity limit the amount of rotation between the two parts, the O-ring cannot simply roll out of a portion of the detent groove and unseal it in that location.

However, if a longitudinal force is applied that attempts to pull coupler body 102 and stalk 104 apart, the smooth surfaces of the nose and inside wall would allow them to slide against each other even under bearing pressure. Thus, a relatively small tensile force could pull apart the safety coupler even if its parts were under bending loads.

The O-ring and detent groove work together as a calibrated unit to allow a relatively small threshold torsional force to pull apart the coupler, and they are able to keep their seal even under torsional forces. The amount that the O-ring radially protrudes from the nose is part of the geometry that keeps the seal.

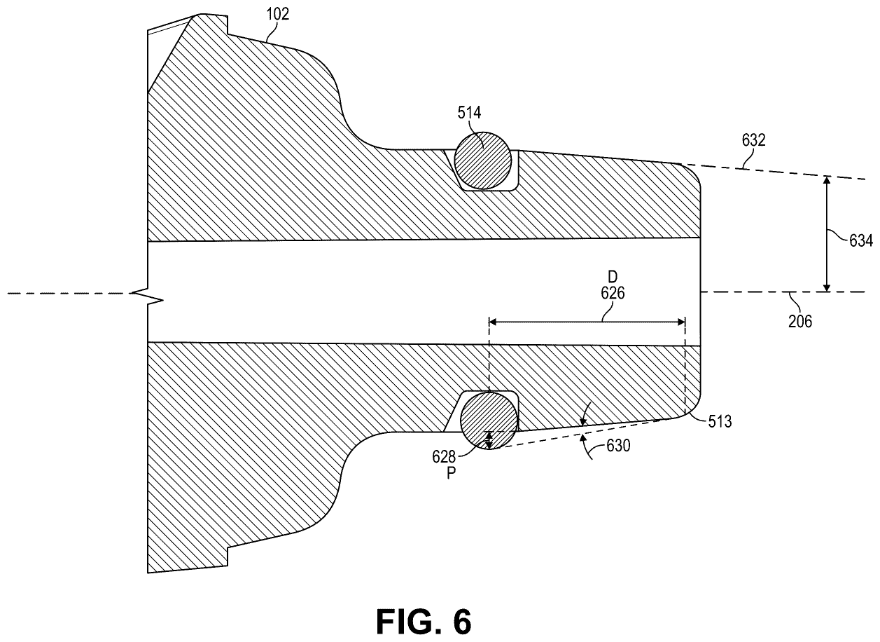

FIG. 6 is a longitudinal cross-section of an unmated coupler body 102. O-ring 514 extends around the circumference of the base of the nose.

The distance from O-ring 514 to nose tip 513 is distance 626, or ‘D.’ The radial distance that the O-ring protrudes out of its trench is protrusion 628, or ‘P.’ The O-ring protrusion P is greater-than-or-equal-to distance D multiplied by the tangent of 15°, 10°, 9°, 8°, 7°, 6°, 5°, 4°, 3°, 2°, 1°, or 0.5°. This angle corresponds to the angle of maximum misalignment that an inside wall, without a detent groove, can be at before the O-ring is no longer compressed against it. This angle of misalignment is the same as that between the longitudinal axes. The previous figures show a misalignment of 2.2°. With typical materials and distances, a protrusion of at least:

$$P = D \cdot tan ( 5 ° ) \qquad ( Equation l )$$

is deemed suitable, even for a slight frustrum nose.

Not to be confused with the angle of misalignment, angle 634 is the angle between longitudinal axis 206 of coupler body 102 and the angle 632 of the conical side of the nose. That is, angle 634 is the angle of the frustum. A zero-degree (0°) angle would be a perfect geometrical cylinder, i.e., not a frustum. A slightly conical frustrum for both the cylindrical nose and the cylindrical cavity allows an end user to more easily align and plug the nose into the cavity.

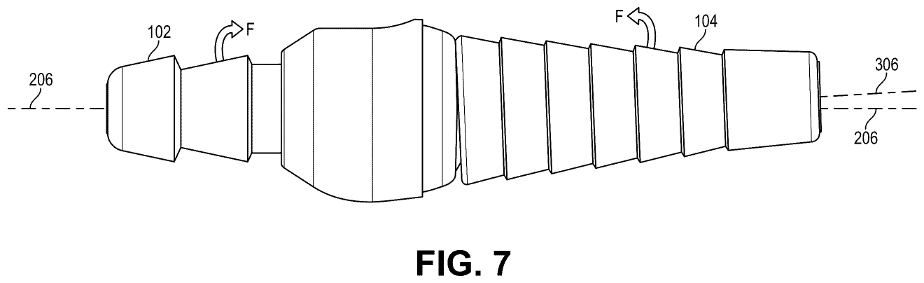

FIG. 7 is a side view of the coupler's constituent coupler body 102 and stalk 104 undergoing bending forces. The misalignment forces cause their longitudinal axes, 206 and 306, respectively, to rotate with respect to each other by an angle, that angle being held small by the bearing surfaces inside.

A technical advantage of the design is that tensile pull-apart forces can be carefully calibrated using an O-ring and detent groove with little regard to bending forces. An O-ring's inner and outer radiuses, along with its material's Poisson's ratio and Young's modulus, are fit to a pre-molded, certain diameter trench seat in the nose so that the O-ring is secure to the nose and under proper tension. The amount that the O-ring radially protrudes from the nose is matched with a depth of a detent groove in the mating stalk. The detent groove's width is sized to keep the O-ring compressed against it and seated all of the way around-up to a given angle. The angle is that which the nose and cavity keep the parts aligned. This can effectively decouple the constraints regarding the relatively soft pull-apart forces from the constraints for extreme force lateral yanking. The O-ring serves both to calibrate the required tensile disconnect force and also to keep a seal. Further, the soft pull-apart forces can be further refined with lubricant on the O-ring.

Although the O-ring can do both duties regarding disconnect force and sealing, more O-rings or other features can be applied.

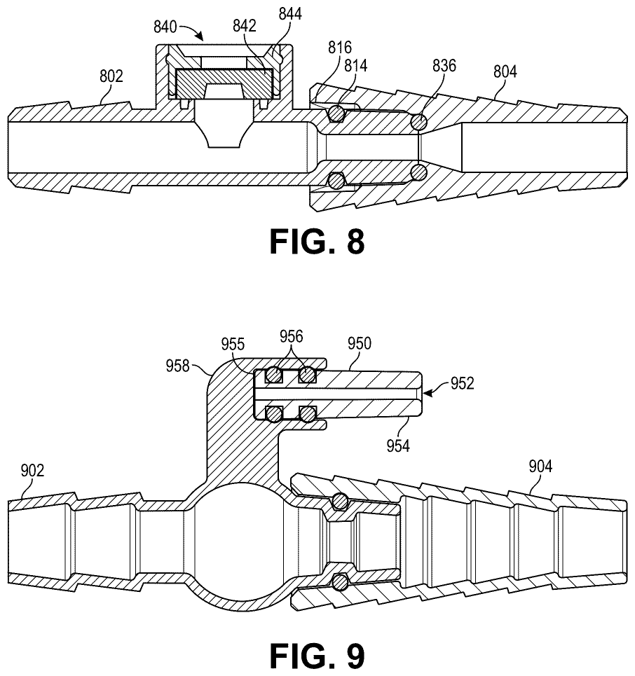

FIG. 8 is a longitudinal cross-section of a coupler body 802 with both a force-calibrated O-ring 814 and a face-sealing O-ring 836. The O-rings 814 and 836 mate against the inside wall of a conical cavity of stalk 804. Instead of protruding radially, face-sealing O-ring 836 protrudes longitudinally from an end of the nose, annularly around the lumen.

The conical cavity includes a stepped portion with an ethylene oxide or autoclave sterilization groove 816. This allows the volume between the disconnect O-ring 814 and the sealing O-ring 836 to be open to sterilization. This is not necessary for gamma- or electron-beam irradiation. However, it has the benefit of removing a vacuum effect in that volume during a disconnection.

Sample port 840 can be used to take samples of fluid running through the coupler. Unlike the sample port shown in FIG. 4, sample port 840 is not recessed in a spherical body but includes a protruding cylinder with a slightly radiused 90 degree edge around its circumference.

Silicone septum 842 sits within plastic septum clip 844. Septum clip 844 is essentially a plastic ring, and it sits within a groove molded into the inner circumference of the sample port. In some embodiments, a split valve port can be used. Other parts can be integrated with the safe disconnect coupler.

FIG. 9 is a longitudinal cross-section of a safe disconnect coupler that has a balloon injection port holster. Coupler body 902 is rigidly attached to holster 958, which is essentially a removable stopper for male luer adapter 950. Male luer adapter 950 has end 954 that is configured to mate with a female injection port of a Foley catheter balloon and, in doing so, keep open a valve in the injection port. Fluid path 952 extends from end 954, through male luer adapter 950, to opposite end 955, where it is stopped up by removable stopper/holster 958.

A set of O-rings keeps the injection port sealed within the cavity of stopper 958 and pressure in the catheter balloon.

If stalk 904 is pulled from coupler body 902, the next to follow would be male luer adapter 950 being pulled from removable stopper 958. Because male luer adapter 958 remains in the injection port, holding its valve open, fluid can drain from the catheter balloon, through the injection port into fluid path 952—and then out end 955. This allows the catheter balloon to deflate, at least a little, before being pulled through a urethra.

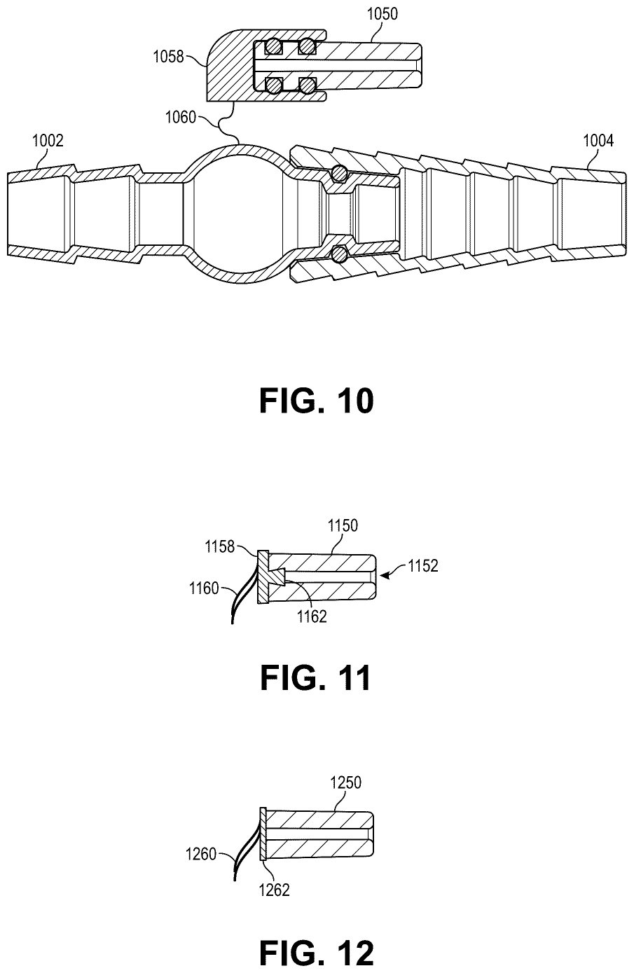

FIG. 10 is a longitudinal cross-section of a coupler with a tethered balloon injection port holster. Instead of a rigid connection, coupler body 1002 is attached by flexible tether 1060 to removable stopper 1058. When male luer adapter 1050 is pulled, it pulls and moves the removable stopper for a little distance until the tether is taut.

The short tether gives a small delay between a disconnection of coupler body 1002 and stalk 1004 and a disconnection of male luer adapter 1050 from its stopper 1058. This delay may prevent minor pullout events of the coupler, which a medical assistant may reconnect, from immediately transitioning to a major pullout event in which a catheter balloon is drained. The latter may need a physician to reconnect and refill.

FIGS. 11 and 12 are longitudinal cross-sections of male luer adapters, each figure showing alternate removable stoppers.

FIG. 11 shows male luer adapter 1150 with removable stopper 1158, which includes plug 1162 that fits into the lumen of the adapter. When tether 1160 is pulled, the plug and removable stopper disengage from the luer, allowing fluid to drain from the catheter balloon.

FIG. 12 shows male luer adapter 1250 with removable stopper 1262 in the form of a burst plate that blocks the fluid path. A burst valve may also be used. When tether 1260 is pulled, burst plate 1262 peels away, allowing fluid to drain from the catheter balloon.

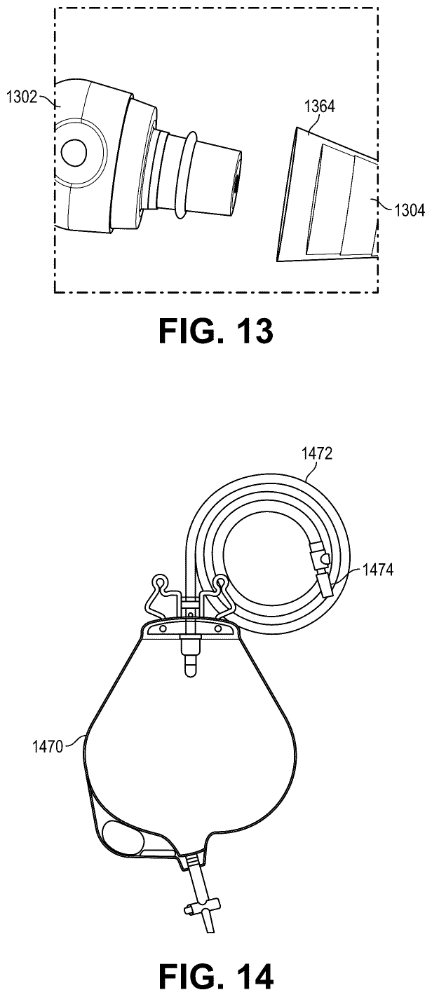

FIG. 13 illustrates a side view of a coupler reconnection prevention mechanism. When coupler body 1302 and stalk 1304 were mated, clear shrink wrap 1364 was formed over the connection. The shrink wrap helps keep the coupler sterile. Then, the coupler body and stalk were pulled apart.

The figure shows coupler body 1304 and stalk 1304 after they were disconnected. Shrink wrap 1364 fouls the end of stalk 1304, preventing it from being cleanly reconnected to coupler body 1302. Other mechanisms to prevent reconnection are envisioned, such as breakaway tabs or pins.

FIG. 14 illustrates a urine drainage bag 1470 ready to be connected to a coupler. Drainage bag 1470 is connected by medical tubing 1472 to safe disconnect coupler 1474. It is at coupler 1474 where a catheter hub would mate, sending two tubes to the patient: a catheter drainage tube and a catheter balloon fill tube.

The coupler body can include a rounded reducer section and an outer profile with no hooked elements or sharp protrusions. For example, a sample port may be rounded and convex on its exterior so as to go from a large diameter to the small diameters of the medical tubing on each side.

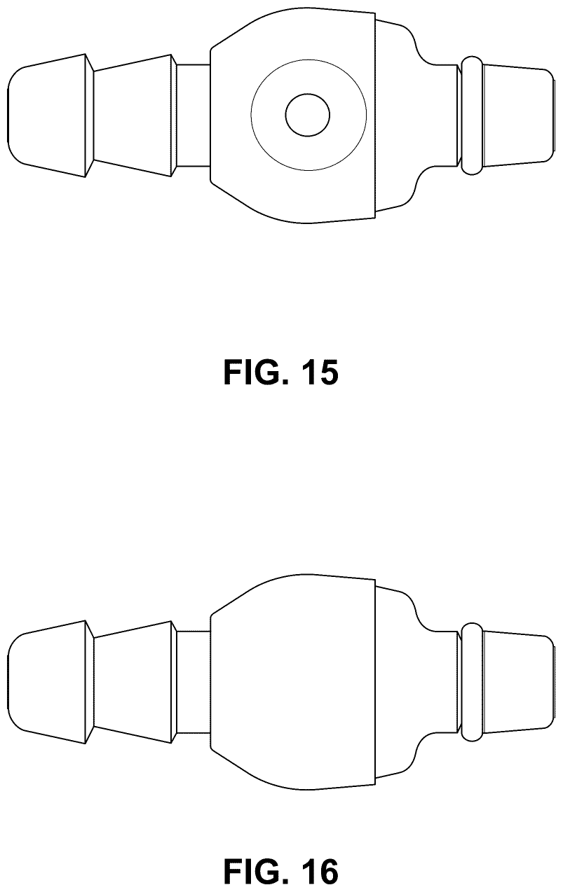

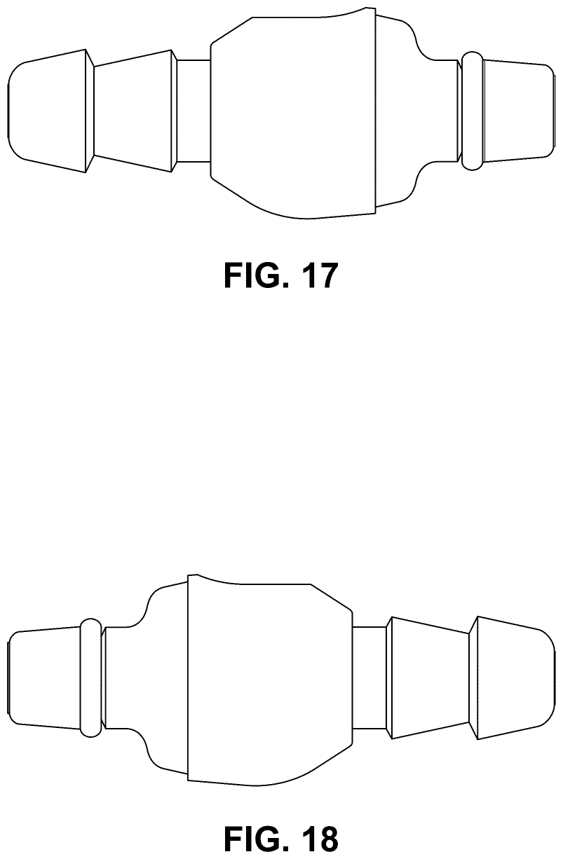

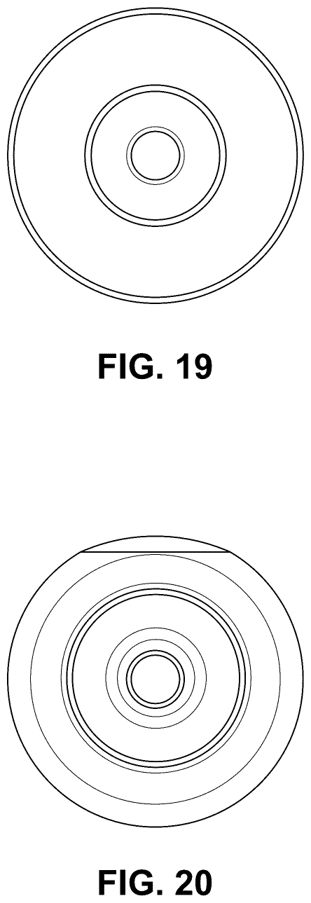

FIGS. 15-20 show views of a coupler body that includes two rounded reducer sections, one on each side of a large diameter port. It has no hooked elements or sharp protrusions that can snag on hospital bedding, furniture, or other implements.

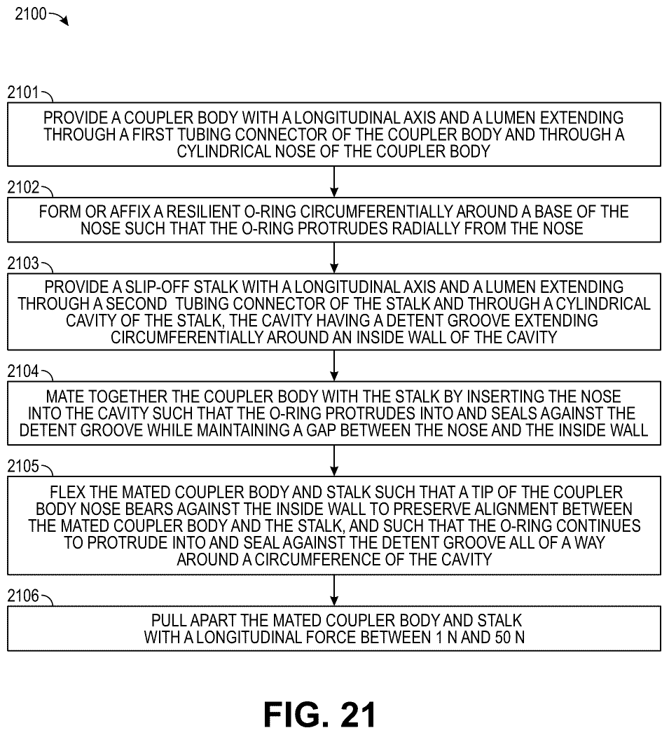

FIG. 21 is a flowchart illustrating process 2100 in accordance with an embodiment. In operation 2101, a coupler body with a longitudinal axis and a lumen extending through a first tubing connector of the coupler body and through a cylindrical nose of the coupler body is provided. In operation 2102, a resilient O-ring is formed or affixed circumferentially around a base of the nose such that the O-ring protrudes radially from the nose. In operation 2103, a slip-off stalk with a longitudinal axis and a lumen extending through a second tubing connector of the stalk and through a cylindrical cavity of the stalk is provided, the stalk having a detent groove extending circumferentially around an inside wall of the cavity. In operation, 2104, the coupler body is mated together with the stalk by inserting the nose into the cavity such that the O-ring protrudes into and seals against the detent groove while maintaining a gap between the nose and the inside wall. In operation 2105, the mated coupler body and stalk are flexed such that a tip of the coupler body nose bears against the inside wall to preserve alignment between the mated coupler body and the stalk, and such that the O-ring continues to protrude into and seal against the detent groove all of a way around a circumference of the cavity. In operation 2106, the mated coupler body and stalk are pulled apart with a longitudinal force between 1 N and 50 N.

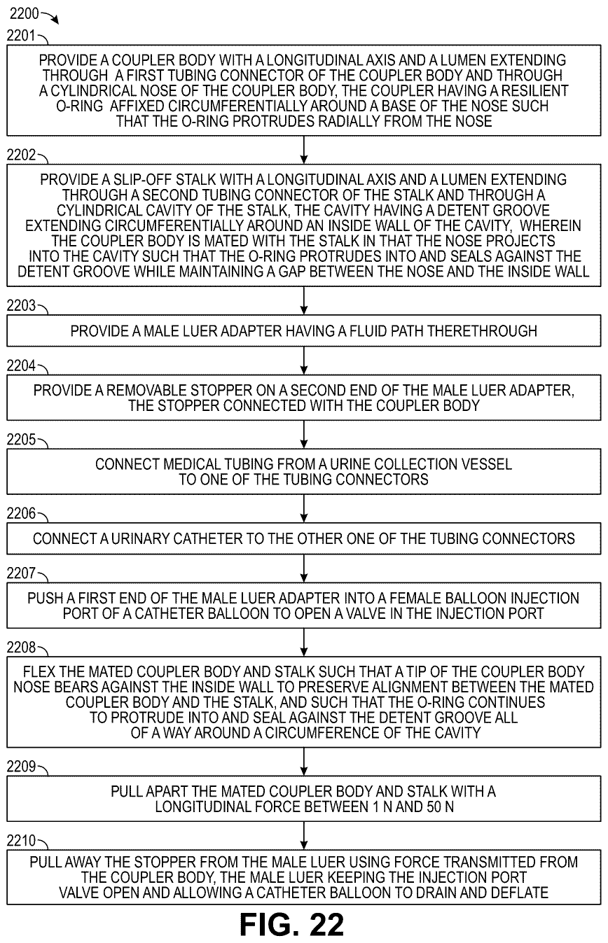

FIG. 22 is a flowchart illustrating process 2200 in accordance with an embodiment. In operation 2201, a coupler body with a longitudinal axis and a lumen extending through a first tubing connector of the coupler body and through a cylindrical nose of the coupler body is provided, the coupler having a resilient O-ring affixed circumferentially around a base of the nose such that the O-ring protrudes radially from the nose. In operation 2202, a slip-off stalk with a longitudinal axis and a lumen extending through a second tubing connector of the stalk and through a cylindrical cavity of the stalk is provided, the cavity having a detent groove extending circumferentially around an inside wall of the cavity, wherein the coupler body is mated with the stalk in that the nose projects into the cavity such that the O-ring protrudes into and seals against the detent groove while maintaining a gap between the nose and the inside wall. In operation 2203, a male luer adapter having a fluid path therethrough is provided. In operation 2204, a removable stopper is provided on a second end of the male luer adapter, the stopper connected with the coupler body. In operation 2205, medical tubing from a urine collection vessel is connected to one of the tubing connectors. In operation 2206, a urinary catheter is connected to the other one of the tubing connectors. In operation 2207, a first end of the male luer adapter is pushed into a female balloon injection port for a catheter balloon in order to open a valve in the injection port. In operation 2208, the mated coupler body and stalk are flexed such that a tip of the coupler body nose bears against the inside wall to preserve alignment between the mated coupler body and the stalk, and such that the O-ring continues to protrude into and scal against the detent groove all of a way around a circumference of the cavity. In operation 2209, the mated coupler body and stalk are pulled apart with a longitudinal force between 1 N and 50 N. In operation 2210, the stopper is pulled away from the male luer using force transmitted from the coupler body, the male luer keeping the injection port valve open and allowing a catheter balloon to drain and deflate.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. “About” in reference to a temperature or other engineering units includes measurements or settings that are within ±1%, ±2%, ±5%, ±10%, or other tolerances of the specified engineering units as known in the art.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A safe disconnect coupler apparatus for medical tubing, the apparatus comprising:
a coupler body with a longitudinal axis and a lumen extending through a first tubing connector of the coupler body and through a cylindrical nose of the coupler body;
a resilient O-ring circumferentially surrounding a base of the nose and protruding radially from the nose; and
a slip-off stalk with a longitudinal axis and a lumen extending through a second tubing connector of the stalk and through a cylindrical cavity of the stalk, the cavity having a detent groove extending circumferentially around an inside wall of the cavity,
wherein the coupler body and the stalk are configured to mate together by inserting the nose into the cavity such that the O-ring protrudes into and seals against the detent groove while maintaining a gap between the nose and the inside wall;
wherein when misalignment forces are applied to the mated coupler body and the stalk, a tip of the coupler body nose bears against the inside wall of the stalk to preserve alignment 14 between the mated coupler body and the stalk, and the O-ring continues to protrude into and seal against the detent groove all of a way around a circumference of the cavity.

2. The apparatus of claim 1 wherein the O-ring protrusion, a material of the O-ring, and a shape of the detent groove are selected such that a mated coupler body and stalk are configured to separate upon a longitudinal pull-apart force between 1 N and 50 N.

3. The method of claim 1 wherein given a distance D from the tip of the coupler body nose to the O-ring, the O-ring protrudes from the coupler body by at least) Dtan(5°) when uncompressed.

4. The apparatus of claim 1 wherein the cylindrical nose and the cylindrical cavity are slightly conical frustums.

5. The apparatus of claim 1 wherein the O-ring is a polymer that is over-molded around the nose.

6. The apparatus of claim 5 wherein the polymer is selected from the group consisting of a silicone, a fluoroelastomer, an acrylonitrile butadiene rubber, a polypropylene, and a polyethylene.

7. The apparatus of claim 1 wherein the O-ring is a distinct part that is held within a trench seat in the nose.

8. The apparatus of claim 1 wherein the O-ring is a first O-ring, the apparatus further comprising:
a second O-ring on the nose and configured to seal against the inside wall.

9. The method of claim 1 further comprising:
a lubricant or lubricious polymer on the O-ring.

10. The method of claim 1 wherein the coupler body includes a rounded reducer section and an outer profile with no hooked elements or sharp protrusions.

11. The method of claim 1 further comprising:
a sample port in the coupler body, the sample port comprising a septum configured to allow needle access inside the coupler body.

12. The apparatus of claim 1 further comprising:
a male luer adapter having a fluid path therethrough and a first end configured to mate with a female balloon injection port of a catheter balloon and keep open a valve in the injection port; and
a removable stopper on a second end of the male luer adapter, the stopper connected with the coupler body,
wherein the stopper is configured to seal the fluid path until pulled away from the male luer adapter by force transmitted from the coupler body.

13. The apparatus of claim 12 further comprising:
a tether connecting the removable stopper to the coupler body.

14. The apparatus of claim 12 wherein the removable stopper is rigidly connected with the coupler body.

15. The apparatus of claim 1 wherein the nose of the coupler body is mated with the cavity of the stalk.

16. The apparatus of claim 15 further comprising:
a reconnection prevention mechanism configured to prevent the coupler body and cavity from being mated again after the coupler body and cavity are pulled apart.

17. A urinary catheter urine collection kit comprising:
a urine collection vessel;
a catheter hub; and
the safe disconnect coupler apparatus of claim 1.

18. A method of manufacturing a safe disconnect coupler for medical tubing, the method comprising:
providing a coupler body with a longitudinal axis and a lumen extending through a first tubing connector of the coupler body and through a cylindrical nose of the coupler body;
forming or affixing a resilient O-ring circumferentially around a base of the nose such that the O-ring protrudes radially from the nose;
providing a slip-off stalk with a longitudinal axis and a lumen extending through a second tubing connector of the stalk and through a cylindrical cavity of the stalk, the cavity having a detent groove extending circumferentially around an inside wall of the cavity; and
mating together the coupler body with the stalk by inserting the nose into the cavity such that the O-ring protrudes into and seals against the detent groove while maintaining a gap between the nose and the inside wall.

19. The method of claim 18 including testing, the method further comprising:
flexing the mated coupler body and stalk such that a tip of the coupler body nose bears against the inside wall to preserve alignment between the mated coupler body and the stalk, and such that the O-ring continues to protrude into and seal against the detent groove all of a way around a circumference of the cavity.

20-25. (canceled)

26. A safe disconnect urinary catheter collection vessel apparatus comprising:
a urine collection vessel;
a medical tubing connected with the vessel; and
a safe disconnect coupler connected to an end of the medical tubing, the coupler being configured to separate into a coupler body and another part upon a longitudinal pull-apart force between 1 N and 50 N.

27. The apparatus of claim 26 further comprising:
a male luer adapter having a fluid path therethrough and a first end configured to mate with a female balloon injection port of a catheter balloon and keep open a valve in the injection port;
a removable stopper on a second end of the male luer adapter, the stopper connected with the coupler body,
wherein the stopper is configured to seal the fluid path until pulled away from the male luer adapter by force transmitted from the coupler body.

28. The apparatus of claim 27 further comprising:
a flexible tether connecting the removable stopper to the coupler body.

29. The apparatus of claim 27 wherein the removable stopper is rigidly connected with the coupler body.

30. The method of claim 27 further comprising:
a female balloon injection port mated with the male luer adapter.

31. The method of claim 26 further comprising:
a urinary catheter connected with the coupler.
